(19) United States
(12) Patent Application Publication
(10) Pub. No.: US 2022/0079925 A1
(43) Pub. Date: Mar. 17, 2022

(54) COMPOSITIONS COMPRISING TACROLIMUS FOR THE TREATMENT OF INTRAOCULAR INFLAMMATORY EYE DISEASES

(71) Applicant: NOVALIQ GMBH, Heidelburg (DE)

(72) Inventors: Bernhard GÜNTHER, Dossenheim (DE); Dieter SCHERER, Laufen (CH); Heping XU, Belfast (GB)

(21) Appl. No.: 17/486,634

(22) Filed: Sep. 27, 2021

Related U.S. Application Data
(63) Continuation of application No. 16/472,831, filed on Jun. 21, 2019.

(30) Foreign Application Priority Data
Dec. 22, 2016 (EP) 16206207.9

(51) Int. Cl. A61K 31/436; A61P 27/02; A61K 9/00; A61K 47/06

(52) U.S. Cl. CPC A61K 31/436; A61K 47/06; A61K 9/0048; A61P 27/02

(57) ABSTRACT

The present invention relates to a pharmaceutical composition comprising a therapeutically effective amount of tacrolimus and a liquid vehicle comprising at least one semifluorinated alkane for use in a method of treating an intraocular inflammatory eye disease.

### CROSS-REFERENCE TO RELATED APPLICATIONS

This application is a U.S. national stage application filed under 35 U.S.C. § 371 of International Application No. PCT/EP2017/082739, filed on Dec. 14, 2017, which claims priority to, and the benefit of, European Application No. 16206207.9, filed Dec. 22, 2016, the contents of each of which are incorporated herein by reference in their entireties.

### Description

### BACKGROUND OF THE INVENTION

Inflammatory eye diseases comprise various types of inflammation that may affect any part of the eye or the surrounding tissue. The broad range of known inflammatory eye diseases comprises, for example, very common conditions such as allergic conjunctivitis due to hay fever or more rare and dangerous conditions such as uveitis, scleritis, optic neuritis, keratitis, retinal vasculitis or chronic vasculitis, all of which have the potential to endanger the eyesight of an affected person.

Some inflammatory eye diseases are effectively treated with corticosteroids such as, for example, dexamethasone. Long-term use of dexamethasone, however, may result in severe side effects as commonly known from long-term treatment with corticosteroids in general. Common side effect associated with the administration of corticosteroids such as dexamethasone is the development of a cataract or glaucoma. The increased risk of the development of a cataract or glaucoma would be especially problematic in the case of the treatment of an already existing inflammatory eye disease.

Uveitis is an inflammatory condition of the uveal tract of the eye. Clinically, it can be classified into anterior, intermediate, posterior and pan uveitis based on the part of the uveal tract affected. Anterior uveitis accounts for 60 to 80% of all uveitis cases. Uveitis remains a significant cause of blindness in people of working age accounting for 10 to 15% of total blindness in the US.

Uveitis can have infectious or non-infectious (autoimmune) etiologies. Autoimmune uveitis is mediated by retinal antigen specific T lymphocytes, including Th1 and Th17 cells, and corticosteroids remain the mainstay therapy for this condition.

Anterior uveitis is usually treated with topical steroids, although systemic immune suppression may be required if patients also have other systemic autoimmune conditions. For posterior uveitis, apart from systemic immune suppression, intravitreal injection of steroids (e.g., dexamethasone, triamcinolone and fluocinolone) are now commonly used.

Various intraocular implants have also been developed to improve intraocular drug delivery. Although intravitreal injection of steroids can better control inflammation and cause limited systemic side-effects, local adverse effects remain a major concern. For example, 30 to 50% of patients developed glaucoma following intravitreal injection of triamcinolone; almost all patients received intravitreal injections of fluocinolone via the Retisert implant had glaucoma and 40% of them required surgery to control the pressure. Cataract is another common side-effect, particularly in patients receiving multiple intravitreal steroid injections. Clearly, more effective and safer therapies are urgently needed for uveitis.

Tacrolimus (FK506), a macrolide lactone isolated from fungus Streptomyces tsukubaensis is a potent immunosuppressive drug. Tacrolimus has the same mechanistic action of inhibiting T-lymphocyte signal transduction and cell proliferation like cyclosporine, but is 100 times more powerful. Tacrolimus has been reported to be used as a second line of therapy for uveitis through systemic administration and is proved to be effective, particularly in steroid-resistance or intolerance patients.

Tacrolimus has a poor ability to penetrate tissue barrier upon topical administration due to its physicochemical properties (Tamura et al., 2002, J. Pharm. Sci. 91, 719-729).

US 2003/0018044 A1 describes a formulation containing Tacrolimus to treat an ocular disease such as dry eye disease (DED), as well as other eye diseases. Tacrolimus may be dissolved in an aqueous solvent such as 0.9% saline or 5% dextrose, or an organic solvent such as dimethylsulfoxide (DMSO) or an alcohol.

WO 2011/073134 A1 provides pharmaceutical compositions for the treatment of keratoconjunctivitis sicca comprising liquid vehicles which include one or more semifluorinated alkanes. The compositions incorporate an active ingredient selected from the group of macrolide immunosuppressants. They can be administered topically into the eye.

WO 2012/160179 A2 describes liquid or semi-solid pharmaceutical compositions for topical administration comprising a semifluorinated alkane. The compositions are useful for the delivery of active ingredients into deep layers of the skin or skin appendages. Various active ingredients may be incorporated, such as immunosuppressants, antiinfectives, antifungal agents, anti-inflammatory agents, and retinoids.

WO 2012/160180 A2 describes semi-solid or liquid pharmaceutical compositions for topical administration to a finger- or toenail of a human. The compositions are useful for the delivery of active ingredients deep into the nail. Various active ingredients may be incorporated, such as antifungal agents, anti-infectives, anti-inflammatory agents immunosuppressants, local anaesthetics, and retinoids.

It is an object of the present invention to provide a pharmaceutical composition that is useful and effective in the treatment of a broad range of intraocular inflammatory eye diseases and that may be administered topically.

A further object of the present invention is to provide a pharmaceutical composition that allows for safe handling, precise dosing and a convenient and an easy to follow dosage regime to support regular administration and overall compliance by the patient in need of such treatment.

It is a further object of the invention to provide a pharmaceutical composition for use in the treatment of intraocular inflammatory eye diseases useful and effective in addition or as a substitute to common first-line treatments of such eye diseases and which does not show the side effects of standard steroid treatments with regard to increase of intraocular pressure.

### SUMMARY OF THE INVENTION

The present invention provides a pharmaceutical composition comprising a therapeutically effective amount of tacrolimus and a liquid vehicle comprising at least one semifluorinated alkane for use in a method of treating an intraocular inflammatory eye disease.

The present invention further provides a kit comprising a pharmaceutical composition comprising a pharmaceutically effective amount of tacrolimus and a liquid vehicle comprising at least one semifluorinated alkane for use in a method for the treatment of an intraocular inflammatory eye disease; and a container for holding the composition, wherein the container preferably comprises dispensing means adapted for topical administration of the composition to an eye surface, preferably into a lower eyelid, to the lacrimal sac or to an ophthalmic tissue.

### BRIEF DESCRIPTION OF THE DRAWINGS

FIG. 1A to FIG. 1F: Effects of tacrolimus/SFA eyedrop treatment on clinical presentations of Endotoxin induced uveitis (EIU) mouse model.

FIG. 2A to FIG. 2E: Histopathology of EIU in different groups of mice.

FIG. 3A to FIG. 3F: Effects of tacrolimus/SFA eyedrop treatment on clinical presentation of Experimentally autoimmune uveoretinitis (EAU).

FIG. 4A to FIG. 4E: Histopathology of EAU in different groups of mice.

FIG. 5A to FIG. 5D: Tacrolimus levels in the vitreous (FIG. 5A), choroid/sclera (FIG. 5B), retina (FIG. 5C) and whole blood (FIG. 5D) of normal mouse eyes.

FIG. 6A to FIG. 6D: Tacrolimus levels in the vitreous of mouse eyes with and without uveitis.

FIG. 7: Infiltrating immune cells in retina using flow cytometry in EIU studies.

### DETAILED DESCRIPTION OF THE INVENTION

In a first aspect, the present invention relates to a pharmaceutical composition comprising a therapeutically effective amount of tacrolimus and a liquid vehicle comprising at least one semifluorinated alkane for use in a method of treating an intraocular inflammatory eye disease.

The pharmaceutical composition of the present invention comprises tacrolimus in a therapeutically effective amount. Tacrolimus (also known as FK-506 or fujimycin; CAS 104987-11-3) is a macrolide lactone with the formula C₄₄H₆₉NO₁₂ and a molecular mass of 804,018 g/mol.

Tacrolimus may be isolated from the fermentation broth of the bacterium Streptomyces tsukubaensis and is commercially available, e.g. from Zhejian Hisun Chemical Co, China, and is usually supplied in form of its solid monohydrate or in form of a stock solution. The term “therapeutically effective amount” of tacrolimus as used herein means a dose, concentration or strength of tacrolimus which is useful for producing the desired pharmacological effect, more specifically the pharmacological effect in the treatment of an intraocular inflammatory eye disease as described in detail below.

In a preferred embodiment, the pharmaceutical composition of the present invention is a liquid preparation. The term “liquid preparation” as used herein means any preparation that is not entirely solid. The liquid preparation according to the present invention may be free flowing liquids or in other words have a low viscosity or may be liquids with a medium or high viscosity such as a cream or a paste or a gel. Preferably, however the present pharmaceutical compositions are free flowing liquids, at least at physiological temperature, which may be administered dropwise.

The pharmaceutical composition of the present invention may be formulated in form of a solution, suspension or emulsion. Preferably, the pharmaceutical composition of the present invention is formulated as a solution, even more preferred as a clear solution.

The term a “clear solution”, as mentioned above and understood herein, refers to a liquid solution in which all solutes are fully dissolvable or dissolved under room temperature conditions i.e. between 15 and 25° C. The clear solution does not comprise of any particulate or solid phase components and preferably has a refractive index approximate to that of water (i.e. 1.333) at room temperature.

Accordingly, tacrolimus as described above may be dissolved or suspended or emulsified in the liquid vehicle of the present pharmaceutical composition. The term “liquid vehicle” as used herein means the continuous (or coherent) phase, typically the solvent or mixture of different solvents of the present pharmaceutical composition in which tacrolimus is dissolved or suspended or emulsified.

Some of the key advantages of the pharmaceutical composition of the present invention are brought about by the presence of a semifluorinated alkane in the liquid vehicle of the present pharmaceutical compositions, functioning as a liquid suspension vehicle or solvent in case of a solution.

The term “semifluorinated alkane” (also referred to as “SFA” throughout this document) as used herein refers to a linear or branched compound composed of at least one perfluorinated segment (F-segment) and at least one non-fluorinated hydrocarbon segment (H-segment). More preferably, the semifluorinated alkane is a linear or branched compound composed of one perfluorinated segment (F-segment) and one non-fluorinated hydrocarbon segment (H-segment). Preferably, said semifluorinated alkane is a compound that exists in a liquid state at least at one temperature within the temperature range of 4° to 40° C. In one embodiment, the perfluorinated segment and/or the hydrocarbon segment of the said SFA optionally comprises or consists of a cyclic hydrocarbon segment, or optionally said SFA comprises an unsaturated moiety within the hydrocarbon segment.

Preferably, the F-segment of a linear or branched SFA comprises between 3 to 10 carbon atoms. It is also preferred that the H-segment comprises between 3 to 10 carbon atoms. It is particularly preferred that the F- and the H-segment comprise, but independently from one another, 3 to 10 carbon atoms. Preferably, each segment independently from another is having 3 to 10 carbon atoms.

It is further preferred, that the F-segment of a linear or branched SFA comprises between 4 to 10 carbon atoms and/or that the H-segment comprises between 4 to 10 carbon atoms. It is particularly further preferred that the F- and the H-segment comprise, but independently from one another, 4 to 10 carbon atoms. Preferably, each segment is independently from another having 4 to 10 carbon atoms.

Accordingly, in a preferred embodiment of the present invention the liquid vehicle comprises at least one semifluorinated alkane which is a compound, preferably a linear compound, of the formula F(CF₂)ₙ(CH₂)ₘH, wherein n and m are integers independently selected from the range of 3 to 10, preferably selected from the range of 4 to 10 and even more preferably selected from the range of 4 to 8 carbon atoms.

Optionally, the linear or branched SFA may comprise a branched non-fluorinated hydrocarbon segment comprising one or more alkyl groups selected from the group consisting of —CH₃, —C₂H₅, —C₃H₇ and —C₄H₉ and/or the linear or branched SFA may comprise a branched perfluorinated hydrocarbon segment, comprising one or more perfluorinated alkyl groups selected from the group consisting of —CF₃, —C₂F₅, —C₃F₇ and —C₄F₉. It is further preferred that the ratio of the carbon atoms of the F-segment and the H-segment (said ratio obtained by dividing the number of carbon atoms in the F-segment by the numbers of carbon atoms in the H-segment; e.g. said ratio is 0.75 for 1-perfluorohexyloctane (F6H8)) of a linear or branched SFA is ≥0.5, more preferably said ratio is ≥0.6. It is further preferred that the ratio of the carbon atoms of the F-segment and the H-segment is in the range between 0.6 and 3.0, more preferably said ratio is between 0.6 and 1.0.

In a preferred embodiment of the present invention the semifluorinated alkane refers to a linear compound composed of at least one perfluorinated segment (F-segment) and at least one hydrocarbon segment (H-segment). More preferably, said semifluorinated alkane is a linear compound composed of one perfluorinated segment (F-segment) and one hydrocarbon segment (H-segment).

Preferably, the F-segment of a linear SFA comprises between 3 to 10 carbon atoms. It is also preferred that the H-segment comprises between 3 to 10 carbon atoms. It is particularly preferred that the F- and the H-segment comprise, but independently from one another, 3 to 10 carbon atoms. Preferably, each segment independently from another is having 3 to 10 carbon atoms.

It is further preferred, that the F-segment of a linear SFA comprises between 4 to 10 carbon atoms and/or that the H-segment comprises between 4 to 10 and even more preferably 4 to 8 carbon atoms. It is particularly further preferred that the F- and the H-segment comprise, but independently from one another, 4 to 10 carbon atoms, even more preferably 4 to 8 carbon atoms. Preferably, each segment is independently from another having 4 to 10, preferably 4 to 8 carbon atoms.

According to another nomenclature, the linear semifluorinated alkanes as used in the present invention may be referred to as FnHm, wherein F means the perfluorinated hydrocarbon segment, H means the non-fluorinated hydrocarbon segment and n, m is the number of carbon atoms of the respective segment. For example, F4H5 is used for 1-perfluorobutyl pentane.

In a particularly preferred embodiment of the present invention, the liquid vehicle of the pharmaceutical composition for use according to the present invention comprises at least one linear semifluorinated alkane selected from the group consisting of: F4H4, F4H5, F4H6, F4H7, F4H8, F5H4, F5H5, F5H6, F5H7, F5H8, F6H2, F6H4, F6H6, F6H7, F6H8, F6H9, F6H10, F6H12, F8H8, F8H10, F8H12 and F10H10.

More preferably the liquid vehicle of the pharmaceutical composition according to the present invention comprises at least one linear semifluorinated alkane selected from the group consisting of: F4H4, F4H5, F4H6, F5H4, F5H5, F5H6, F5H7, F5H8, F6H2, F6H4, F6H6, F6H7, F6H8, F6H9, F6H10, F8H8, F8H10, F8H12 and F10H10, even more preferably the linear SFA is selected from the group consisting of: F4H4, F4H5, F4H6, F5H4, F5H5, F5H6, F5H7, F5H8, F6H4, F6H6, F6H7, F6H8, F6H9, F6H10, F8H8, F8H10, F8H12 and F10H10, more preferably the linear SFA is selected from the group consisting of: F4H4, F4H5, F4H6, F5H5, F5H6, F5H7, F5H8, F6H6, F6H7, F6H8, F6H9, F6H10, F8H8, F8H10, F8H12 and F10H10 and even more preferably the linear SFA is selected from the group consisting of: F4H4, F4H5, F4H6, F5H5, F5H6, F5H7, F5H8, F6H6, F6H7, F6H8, F6H9, F6H10 and F8H8.

In yet a further preferred embodiment, the liquid vehicle of the pharmaceutical composition according to the present invention comprises at least one linear SFA selected from the group consisting of: F4H5, F4H6, F5H6, F5H7, F6H6, F6H7 and F6H8.

In an even further preferred embodiment the liquid vehicle according to the present invention comprises at least one linear SFA, preferably at least one linear SFA selected from the group consisting of F4H5, F4H6 and F6H8. Even more preferably the liquid vehicle according to the present invention comprises at least one linear SFA selected from the group consisting of F4H5 and F6H8. Most preferably in the present invention the liquid vehicle comprises F4H5 (1-perfluorobutyl-pentane) as the only semifluorinated alkane.

In a further embodiment, the semifluorinated alkane comprised in the liquid vehicle may be used as a mixture of two or more different semifluorinated alkanes as described above. Accordingly, the present pharmaceutical composition may comprise more than one SFA. It may be useful to combine different SFA's, for example, in order to achieve a particular target property such as a certain density or viscosity. If a mixture of two or more different SFA's is used, it is furthermore preferred that the mixture comprises at least one of F4H5, F4H6, F6H4, F6H6, F6H8 and F6H10, and in particular one of F4H5, F6H6 and F6H8. In another embodiment, the mixture comprises at least two members selected from F4H5, F4H6, F6H4, F6H6, F6H8, and F6H10, and in particular at least two members selected from F4H5, F6H6 and F6H8.

Liquid SFA's are chemically and physiologically inert, colourless and stable. Their typical densities range from 1.1 to 1.7 g/cm³, and their surface tension may be as low as 19 mN/m. SFA's of the FnHm type are insoluble in water but also somewhat amphiphilic, with increasing lipophilicity correlating with an increasing size of the non-fluorinated segment.

In another embodiment, the liquid vehicle of the pharmaceutical composition of the present invention comprises at least one semifluorinated alkane that is liquid at room temperature, such as F4H4, F4H5, F4H6, F5H5, F5H6, F5H7, F5H8, F6H6, F6H7, F6H8, F6H9, F6H10 and F8H8.

A particularly preferred semifluorinated alkane comprised by the liquid vehicle of the pharmaceutical composition of the present invention is 1-perfluorobutyl-pentane, a semifluorinated alkane with the chemical formula F(CF₂)₄(CH₂)₅H. It is an inert, water-insoluble liquid, with a density of 1.284 g/cm³ at 25° C. and refractive index of 1.3204 at 20° C. Alternative nomenclature for this compound includes F4H5, wherein F denote a linear perfluorinated alkane segment comprising 4 carbon atoms and wherein H denotes a linear and non-fluorinated alkane hydrocarbon segment of 5 carbon atoms.

The pharmaceutical composition for use according to the present invention comprises tacrolimus in a therapeutically effective amount, and a liquid vehicle comprising at least one semifluorinated alkane, whereas in one embodiment the pharmaceutical composition of the present invention comprises or consists of at least 75% wt.-%, preferably of at least 80 wt.-%, more preferably of at least 85 wt.-%,even more preferably of at least 90 and most preferably of at least 95 wt.-% of the liquid vehicle comprising at least one semifluorinated alkane, based on the weight of the final pharmaceutical composition (final dosage form). The pharmaceutical composition comprising tacrolimus and a liquid vehicle comprising at least one semifluorinated alkane may optionally comprise further solvents and excipients as described in detail below.

In a preferred embodiment, the pharmaceutical composition of the present invention consists of from about 95 to about 99,999% wt.-%, more preferably from about 95 to about 99.99% wt.-%, more preferably from 99 to 99.99% wt.-% even more preferably from 99.95 to 99.99% wt.-% and most preferred from about 99.96 to about 99.98% wt.-% of the liquid vehicle comprising at least one semifluorinated alkane as described above, based on the weight of the final composition.

In some preferred embodiments, the pharmaceutical composition for use according to the present invention comprises up to 1 mg/ml tacrolimus, preferably up to 0.5 mg/ml tacrolimus and more preferably up 0.3 mg/ml tacrolimus (based on the volume of the final composition (final dosage form).

Accordingly, preferred embodiments of the pharmaceutical composition for use according to the present invention comprise about 0.01% (w/v) to 0.1% (w/v) (corresponding to 0.1 to 1 mg/ml) of tacrolimus. In a preferred embodiment, however, the present pharmaceutical composition comprises about 0.01% to 0.05% (w/v) (corresponding to 0.1 to 0.5 mg/ml) and even more preferably from about 0.02% to about 0.04% (w/v) (corresponding to 0.2 to 0.4 mg/ml) of tacrolimus.

The pharmaceutical composition of the present invention may also comprise one or more further excipients as an optional and additional component. The term “excipients” as used herein refers to any pharmaceutically acceptable natural or synthetic substance that may be added to the pharmaceutical compositions of the present invention, more specifically to the liquid vehicle of the pharmaceutical composition to enhance or otherwise modify its physical or chemical constitution or stability or therapeutic properties. The present pharmaceutical composition may optionally comprise one or more excipients such as, for example, an antioxidant, a preservative, a lipid or oily excipient, a surfactant or a lubricant or a combination of at least 2 excipients thereof.

Suitable antioxidants for use in the present pharmaceutical composition comprise, for example: butylated hydroxytoluene (BHT), butylated hydroxyanisole (BHA), tertiary butylhydroquinone (TBHQ), vitamin E, vitamin E derivatives (i.e. alpha-tocopherol acetate) and/or ascorbic acid.

Suitable lipid or oily excipients for use in the pharmaceutical composition of the present invention comprise, for example, triglyceride oils (i.e. soybean oil, olive oil, sesame oil, cotton seed oil, castor oil, sweet almond oil), triglycerides, mineral oil (i.e. petrolatum and liquid paraffin), medium chain triglycerides (MCT), oily fatty acids, isopropyl myristate, oily fatty alcohols, esters of sorbitol and fatty acids, oily sucrose esters, or any other oily substance which is physiologically tolerated by the eye.

Suitable lubricants for use in the pharmaceutical composition of the present invention comprise, for example, carboxymethylcellulose and its sodium salt (CMC, carmellose), polyvinyl alcohol, hydroxypropyl methylcellulose (HPMC, hypromellose), hyaluronic acid and its sodium salt, and hydroxypropyl guar gum.

The pharmaceutical composition according to the present invention may or may not comprise pharmaceutically suitable natural or synthetic preservatives, such as, for example, benzalkonium chloride and chlorhexidine. In a preferred embodiment, however, the pharmaceutical composition according to the present invention does not comprise a pharmaceutically acceptable preservative.

In addition to the excipients as described above as optional components the liquid vehicle of the present pharmaceutical composition may also comprise one or more further solvents. The term “further solvents” as used herein refers to a solvent or mixture of two or more different solvents other than the at least one semifluorinated alkane of the liquid vehicle. Suitable further solvents may be chosen from, for example, alcohols, such as ethanol, isopropanol or other further solvent which is physiologically tolerated by the eye.

A preferred solvent is ethanol which may be present in the pharmaceutical composition for use according to the present invention in an amount of up to about 1.4 15 wt.-% (corresponding to 1.4% (w/w)) or less, preferably up to about 1.0 wt.-%, such as, for example from 0.2 to 1.0 wt.-% (corresponding to 0.2% to 1.0% (w/w)) or 0.5 to 1.0 wt.-% (corresponding to 0.5 to 1.0% (w/w)), based on the total weight of the liquid vehicle of the final composition (final dosage form). More preferably, the liquid vehicle of the pharmaceutical composition for use according to the present invention comprises 1.0 wt.-% ethanol (in addition to the at least one semifluorinated alkane as described above). Most preferably, the liquid vehicle of the pharmaceutical composition for use according to the present invention comprise 1.4 wt.-% ethanol (in addition to the at least one semifluorinated alkane as described above).

In a further embodiment, water can also be present in the pharmaceutical composition of the present invention, however, preferably in small amounts of up 1.0 wt.-% or even up to 0.1 wt.-% or less, based on the final composition (final dosage form). In a preferred embodiment, the pharmaceutical composition of the present invention is essentially free of water, whereas the residual water may be attributed to the potential residual water content of tacrolimus. The term ‘essentially’ as used herein means if present then in trace or residual amounts such as to confer no technical advantage or relevance in respect of the object of the invention.

For example, the 1-perfluorobutyl-pentane (F4H5) as a preferred semifluorinated alkane in some embodiments of the present invention does not comprise any water, or has a water content of no more than the maximal solubility of water in 1-perfluorobutyl-pentane, for example, has a water-content of less than 1.6×10⁻⁴ mg/ml as determined by methods known in the art for moisture analysis, such as

Karl-Fischer titration methods.

In another preferred embodiment, the pharmaceutical composition of the present invention is (essentially) water-free and/or preservative free.

In a further preferred embodiment, the pharmaceutical composition for use according to the present invention comprises 0.01% to 0.05% (w/v) of tacrolimus, 0.2 to 1.4% (w/w) ethanol (based on the amount of semifluorinated alkane) and the semifluorinated alkane comprised in the liquid vehicle is selected from F4H5 and F6H8. In a yet further preferred embodiment the pharmaceutical composition for use according to the present invention comprises 0.01% to 0.05% (w/v) of tacrolimus, 0.2 to 1.4% (w/w) ethanol (based on the amount of semifluorinated alkane) and the semifluorinated alkane comprised in the liquid vehicle is F4H5. In a further preferred embodiment, the liquid vehicle of the present composition consists of 0.2 to 1.4% (w/w) ethanol (based on the amount of semifluorinated alkane) and the semifluorinated alkane selected from F4H5 and F6H8, preferably the semifluorinated alkane is F4H5.

In a further preferred embodiment, the pharmaceutical composition of the present invention comprises 0.03% (w/v) of tacrolimus, 1.4% (w/w) ethanol and the semifluorinated alkane is F4H5. In an especially preferred embodiment, the pharmaceutical composition of the present invention essentially consists of 0.03% (w/v) of tacrolimus, 1.4% (w/w) ethanol and F4H5, all based on the weight of the final composition (final dosage form).

In a further preferred embodiment, the pharmaceutical composition of the present invention comprises 0.03% (w/v) of tacrolimus, 1.0% (w/w) ethanol and the semifluorinated alkane is F4H5. In an especially preferred embodiment, the pharmaceutical composition of the present invention essentially consists of 0.03% (w/v) of tacrolimus, 1.0% (w/w) ethanol and F4H5, all based on the weight of the final composition (final dosage form).

According to the first aspect, the present invention is directed to the pharmaceutical composition comprising tacrolimus in a therapeutically effective amount, and a liquid vehicle comprising at least one semifluorinated alkane for use in a method of treating an intraocular inflammatory eye disease. The term “intraocular inflammatory eye disease” as used herein means any kind of inflammation in the middle layer of the eye and in the back of the eye. Specifically, inflammatory eye diseases involving the cornea and conjunctiva are not included in the definition of intraocular inflammatory eye diseases as used herein.

The term “inflammatory eye diseases”, as used herein refers to inflammation affecting any part of the eye or surrounding tissue. Inflammation involving the eye can range from the allergic conjunctivitis of hay fever to rare, potentially blinding conditions such as uveitis, scleritis, optic neuritis, keratitis, retinal vasculitis and chronic conjunctivitis.

In a preferred embodiment, the intraocular inflammatory eye disease is selected from the group consisting of uveitis, retinal inflammation, scleritis, optic neuritis and combinations thereof.

Uveitis as mentioned above and its different types as further defined below are an inflammation inside the eye, i. e. an intraocular inflammatory eye disease, specifically affecting one or more of the three parts of the eye that make up the uvea.

The term “uveitis”, as used herein broadly refers to the inflammation of the uvea, the middle layer of the eye that consists of the iris, ciliary body and choroid. The uvea is sandwiched between the sclera (the white part of the eye) and the nerve tissue (retina) inside of the eye. The type of uveitis usually is classified by where the inflammation occurs in the uvea. Uveitis is generally treated with steroids.

The term “anterior uveitis” as used herein means an inflammation of the iris (iritis) or the iris and the ciliar body (iridocyclitis). The main treatment for iritis is steroid eye drops. The main side effect of this treatment resides in lessening the ability of the eye to fight infections and they may accelerate glaucoma and cataract in certain patients.

The term “intermediate uveitis” as used herein means an inflammation of the area behind the ciliar body and the vitreous jelly.

The term “posterior uveitis” as used herein means an inflammation at the back of the eye, the choroid and the retina.

Furthermore, the term “diffuse uveitis” (also called “panuveitis”) as used herein means an inflammation of all areas of the uvea.

Retinal inflammation is a further example of an intraocular inflammatory eye disease. The term “retinal inflammation” as used herein refers to swelling in the tissue at the back wall inside the eye. Retinal inflammation may be caused by cataract surgery, diabetes, a macular pucker, systemic diseases, trauma, or may not have a discernible cause at all. As used herein, the term “retinal inflammation” further comprises diseases of the retina that have an inflammatory component, such as, for example, diabetic retinopathy and age related macular degeneration (AMD).

A further exemplary intraocular inflammatory eye disease is scleritis. The term “scleritis” as used herein refers to the inflammation of the sclera. The sclera is the hard, white outside coating of the eye that provides rigid structural support to the eye. The treatment of scleritis depends on how severe the inflammation is. People with severe forms of scleritis usually need treatment with steroids.

Yet a further intraocular inflammatory eye disease according to the present invention is optic neuritis. The term “optic neuritis” as used herein refers to the inflammation of the optic nerve. The main treatment usually consists in the administration of corticosteroids. The inflammation of the optic nerve head is called papillitis or intraocular optic neuritis. The term “papilitis” as used herein therefore refers to a specific type of optic neuritis. Furthermore, the inflammation of the orbital portion of the nerve is called retrobulbar optic neuritis or orbital optic neuritis.

The pharmaceutical compositions comprising a therapeutically effective amount of tacrolimus and a liquid vehicle comprising at least one semifluorinated alkane as described above are useful for the therapy or treatment of or for the amelioration of the symptoms associated with an intraocular inflammatory eye disease selected from the group consisting of uveitis, retinal inflammation, scleritis and optic neuritis or combinations thereof.

In a further embodiment, the present invention therefore relates to a pharmaceutical composition comprising a therapeutically effective amount of tacrolimus and a liquid vehicle comprising at least one semifluorinated alkane for use in a method of treating an intraocular inflammatory eye disease selected from the group consisting of uveitis, retinal inflammation, scleritis and optic neuritis or combinations thereof. In a further embodiment of the present invention the intraocular inflammatory eye disease is selected from uveitis, retinal inflammation and combinations thereof. As mentioned above, combinations of the intraocular inflammatory eye diseases are possible. This means that either only one of said intraocular inflammatory eye diseases can occur in a patient at the same time or that two, three or all of the four above-mentioned diseases can occur in a patient at the same time and may be successfully treated with the pharmaceutical composition according to the present invention.

In a preferred embodiment of the present invention the intraocular inflammatory eye disease is uveitis. Accordingly, in one embodiment the present invention refers to a pharmaceutical composition comprising a therapeutically effective amount of tacrolimus and a liquid vehicle comprising at least one semifluorinated alkane for use in a method of treating uveitis.

In a specific embodiment, the present invention therefore refers to a pharmaceutical composition comprising tacrolimus in a therapeutically effective amount, and a liquid vehicle comprising at least one semifluorinated alkane for use in a method of treating an intraocular inflammatory eye disease, preferably an uveitis which is selected from the group consisting of anterior uveitis, intermediate uveitis and posterior uveitis or combinations thereof. In other words, preferred embodiments of the present invention refer to pharmaceutical composition comprising a therapeutically effective amount of tacrolimus and a liquid vehicle comprising at least one semifluorinated alkane for use in a method of treating anterior uveitis and/or intermediate uveitis and/or posterior uveitis.

Uveitis can be acute, when it resolves quickly after treatment, or recurrent, when repeated episodes are separated by gaps of several months, or chronic, when the condition continues long-term or requires long-term medication to control it, for example when the condition lasts for more than three months. Further embodiments of the present invention refer to the pharmaceutical composition for use in a method for treatment of an acute or a chronic intraocular inflammatory eye disease such as, for example uveitis and/or retinal inflammation. Further preferred embodiments of the present invention refer to a pharmaceutical composition comprising a therapeutically effective amount of tacrolimus and a liquid vehicle comprising at least one semifluorinated alkane for use in a method of treating uveitis, wherein the uveitis is chronic or acute.

In yet another embodiment the present invention refers to a pharmaceutical composition as described above for use in a method of treating uveitis, wherein the uveitis is refractory uveitis.

As already noted earlier, all mentioned intraocular inflammatory eye diseases to be treated with the pharmaceutical composition of the present invention may occur alone or together with one or two or more different other intraocular inflammatory eye diseases as described above. In a particularly preferred embodiment, the present invention is directed to a pharmaceutical composition as described above for use in the treatment of uveitis or a specific type of uveitis such as anterior uveitis, intermediate uveitis and/or posterior uveitis in combination with retinal inflammation. It should be noted that all intraocular inflammatory eye diseases that may occur in combination with others may or may not be chronic or acute. For example, a chronic uveitis may occur together with an acute retinal inflammation or vice versa.

The pharmaceutical composition of the present invention is especially useful as an ophthalmic composition, and may preferably be administered topically to the eye, eye lid, eye sac, eye surface and/or to an ophthalmic tissue of a patient. Preferably, however, the pharmaceutical composition of the present invention may be topically administered to an outer surface of an eye of a patient or to an ophthalmic tissue which is readily accessible by the patient or by another person administering the pharmaceutical composition to the eye of the patient in need thereof

The pharmaceutical composition, especially when used as liquid of either low or higher viscosity (usually in the range of 1 to 3.5 mPa s) may advantageously administered in form of drops or by spraying or by injection, such as, for example, intravitreal injection or periocular injection. Most preferably, however, the liquid pharmaceutical composition of the present invention may be administered as drops, more specifically as eyedrops to be administered topically to the eye.

Depending on the extend of the disease or on whether or not both eyes of the patient to be treated are affected, the drops or eyedrops of the present ophthalmic pharmaceutical compositions may be administered to only one eye or to both eyes of the patient. Mainly due to specific physical characteristics of the semifluorinated alkanes comprised in the liquid vehicle of the present pharmaceutical composition, such as, for example, a high density and low surface tension the volume of the droplets usually ranges from about 5 to about 50 μl. This small droplet size usually facilitates the dropwise administration and, moreover, facilitates precise dosage of the pharmaceutical composition of the present invention. Accordingly, the ophthalmic pharmaceutical composition of the present invention is administered as single drops with a volume of about 5 to 50 μl per dose per eye, preferably with a volume of about 8 to 15 μl per dose per eye, more preferably with a volume of about 8 to 12 μl per dose per eye and most preferably with a volume of about 10 μl per dose per eye.

The pharmaceutical composition of the present invention may be administered one time per day or several times per day, usually up to 8 times or up to 5 times per day, preferably in more or less equal intervals. In many cases the pharmaceutical composition of the present invention are administered preferably up to 4 times per day, such as only once daily or two times per day or three or four times per day.

In a preferred embodiment, the pharmaceutical composition of the present invention may be administered to patients treated with first-line treatment of an inflammatory eye disease or preferably even with first-line treatment of an intraocular inflammatory eye disease. The term “first-line treatment” as used herein means a treatment of an inflammatory eye disease or, more specifically, a treatment of an intraocular inflammatory eye disease with an active compound or composition other than the pharmaceutical composition of the present invention comprising tacrolimus and a liquid vehicle comprising an semifluorinated alkane. The administration of the present pharmaceutical composition for use in the treatment of an intraocular inflammatory eye disease according to the present invention may, accordingly, be provided as a second-line therapy or adjuvant therapy or treatment, depending on whether or not the chosen first-line therapy or treatment has been stopped before initialization of the second-line treatment or is still ongoing in the case of an adjuvant therapy or treatment.

According to a preferred embodiment, the pharmaceutical composition of the present invention is administered to patients treated with steroids or corticosteroids such as, for example dexamethasone, triamcinolone and fluocinolone, preferably dexamethasone as the first-line treatment. Such steroidal compounds are commonly used as a first-line treatment for many inflammatory eye diseases, especially many intraocular inflammatory eye diseases such as, for example, uveitis and retinal inflammation as outlined above. Preferably, however, the pharmaceutical composition comprising tacrolimus and at least one liquid vehicle comprising a semifluorinated alkane for the use of the present invention are administered (as a second-line treatment) to patients treated with steroids as the first-line treatment of uveitis.

The pharmaceutical composition of the present invention offers a clinical alternative to the standard treatment with steroids or corticosteroids without, however, inducing an increase of intraocular pressure as a common adverse effect of a treatment with steroids or corticosteroids increasing the risk of further complications such as glaucoma or cataract. Furthermore, the pharmaceutical composition of the present invention offers all advantages of a second-line treatment that may be self-administered topically.

Furthermore, it has been surprisingly found that the pharmaceutical composition of the present invention is effective in reducing the number of infiltrating immune cells into the retina. Therefore, the present invention also relates to a method of reducing the number of infiltrating immune cells in the retina of a patient having an inflammatory eye disease or more specifically an intraocular inflammatory eye disease comprising topically administering a pharmaceutical composition comprising a therapeutically effective amount of tacrolimus and a liquid vehicle comprising at least one semifluorinated alkane. Further, the present invention also relates to a method of reducing the number of infiltrating immune cells in the retina of a patient suffering from a disease that has an inflammatory component involving the retina, such as, for example, diabetic retinopathy and age related macular, comprising topically administering a pharmaceutical composition comprising a therapeutically effective amount of tacrolimus and a liquid vehicle comprising at least one semifluorinated alkane.

In a further aspect, the present invention provides a method of treating an intraocular inflammatory eye disease comprising administering a composition comprising tacrolimus and a liquid vehicle comprising at least one semifluorinated alkane to the eye.

In yet a further aspect, the present invention refers to the use of a pharmaceutical composition comprising a therapeutically effective amount of tacrolimus and a liquid vehicle comprising at least one semifluorinated alkane in a method of treating intraocular inflammatory eye disease.

In yet a further aspect, the present invention refers to the use of a pharmaceutical composition comprising tacrolimus, preferably in a therapeutically effective amount, and a liquid vehicle comprising at least one semifluorinated alkane for the manufacture of a medicament for the treatment of an intraocular inflammatory eye disease.

In yet a further aspect, the present invention relates to a kit comprising
i. the pharmaceutical composition comprising a pharmaceutically effective amount of tacrolimus and a liquid vehicle comprising at least one semifluorinated alkane for use in a method for the treatment of an intraocular inflammatory eye disease; and
ii. a container for holding the composition, wherein the container preferably comprises dispensing means adapted for topical administration of the composition to an eye surface, preferably into a lower eyelid, to the lacrimal sac or to an ophthalmic tissue, and
iii. optionally instructions or directions for use of the pharmaceutical composition in the therapy, treatment, prevention or amelioration of intraocular inflammatory eye diseases or disorders.

According to item i. of this aspect of the invention the pharmaceutical kit comprises a pharmaceutical composition comprising a therapeutically effective amount of tacrolimus as described above for the first aspect of the present invention.

A container as used in connection with item ii. of this aspect of the invention can be provided in any suitable form as a container for single use holding a single dose of the pharmaceutical composition or as a container for multiple uses holding a plurality of single doses. Preferably, the container comprises a dispensing means which allows for dropwise topical administration of the pharmaceutical composition to a surface of the eye of a patient. In one embodiment, the container comprising a dispensing means may be a conventional dropper bottle such as a bottle made of glass or a thermoplastic elastomer with a suitable dispensing means or single-use droppers.

In a further preferred embodiment of this aspect of the invention, the dispensing means comprises a dropper of dimensions such as to dispense droplets having a volume of about 8 to 15 μl, preferably of about 8 to 12 μl, more preferably of about 10 μl. With a small droplet volume, precise dosing to the eye (avoiding over-dosing) can be achieved and an excess amount of discharge of a substantial fraction of the composition from the eye subsequent to administration can be avoided.

In a preferred embodiment, the kit according to this aspect of the invention additionally comprises as an item iii. instructions for use.

Instructions or directions for use of the pharmaceutical composition according to item iii. of this aspect of the invention can be provided in any suitable form such as, for example, as an enclosed label or instruction leaflet in printed or other readable form or on any other suitable data carrier. Alternatively, the directions for use can be provided in electronic or computer readable form, such as a barcode or a QR-code.

The directions according to item iii. can comprise instructions for use of the present pharmaceutical compositions in the therapy, treatment, prevention or amelioration of intraocular inflammatory eye diseases or disorders, preferably in the therapy of an intraocular inflammatory eye disease selected from uveitis, retinal inflammation, scleritis and optic neuritis or combinations thereof.

The following are numbered embodiments comprised by the present invention:
1. A pharmaceutical composition comprising a therapeutically effective amount of tacrolimus and a liquid vehicle comprising at least one semifluorinated alkane for use in a method for the treatment of an intraocular inflammatory eye disease, or
a method of treating an intraocular inflammatory eye disease comprising administering a composition comprising a therapeutically effective amount of tacrolimus and a liquid vehicle comprising at least one semifluorinated alkane to the eye, or
the use of a pharmaceutical composition comprising a therapeutically effective amount of tacrolimus and a liquid vehicle comprising at least one semifluorinated alkane in a method of treating intraocular inflammatory eye disease, or
the use of a pharmaceutical composition comprising tacrolimus, preferably in a therapeutically effective amount, and a liquid vehicle comprising at least one semifluorinated alkane for the manufacture of a medicament for the treatment of an intraocular inflammatory eye disease, or
a kit comprising i. the pharmaceutical composition comprising a therapeutically effective amount of tacrolimus and a liquid vehicle comprising at least one semifluorinated alkane for use in a method for the treatment of an intraocular inflammatory eye disease; and ii. a container for holding the composition, wherein the container preferably comprises dispensing means adapted for topical administration of the composition to an eye surface, preferably into a lower eyelid, to the lacrimal sac or to an ophthalmic tissue,
wherein the intraocular inflammatory eye disease is selected from the group consisting of uveitis, retinal inflammation, scleritis and optic neuritis or combinations thereof
2. An embodiment according to embodiment 1, wherein the intraocular inflammatory eye disease is selected from uveitis, retinal inflammation and combinations thereof.
3. An embodiment according to embodiment 2, wherein the intraocular inflammatory eye disease is uveitis.
4. An embodiment according to any of the preceding embodiments wherein the uveitis is refractory uveitis.
5. An embodiment according to any of the preceding embodiments, wherein the uveitis is anterior uveitis and/or intermediate uveitis and/or posterior uveitis.
6. An embodiment according to any of the preceding embodiments, wherein the uveitis is chronic or acute.
7. An embodiment according to any of the preceding embodiments, wherein the semifluorinated alkane is a compound of the formula F(CF₂)ₙ(CH₂)ₘH wherein n and m are integers independently selected from the range of 3 to 10.
8. An embodiment according to embodiment 7, wherein said semifluorinated alkane is selected from the group consisting of F4H5, F4H6, F6H6, F6H8, preferably wherein said semifluorinated alkane is selected from F4H5, F6H8, more preferably said semifluorinated alkane is F4H5.
9. An embodiment according to any of the preceding embodiments, wherein said composition comprises up to 1 mg/ml tacrolimus preferably up to 0.5 mg/ml tacrolimus, more preferably up 0.3 mg/ml tacrolimus.
10. An embodiment according to any of the preceding embodiments said composition comprises an organic excipient, preferably ethanol, more preferably up to 1.4 wt.-% ethanol.
11. An embodiment according to any of the preceding embodiments the composition is a liquid preparation.
12. An embodiment according to embodiment 11, wherein the liquid preparation is water-free and/or preservative free.
13. An embodiment according to any of the preceding embodiments, wherein said composition is formulated in form of a solution, suspension or emulsion, preferably said composition is formulated as a solution.
14. An embodiment according to any of the preceding embodiments, wherein said composition is topically administered to the eye, eye lid, eye sac, eye surface or to an ophthalmic tissue.
15. An embodiment according to any of the preceding embodiments, wherein the composition is administered in form of drops or by spraying or by injection.
16. An embodiment according to any of the preceding embodiments, wherein said composition is administered as single drops with a volume of about 5 to 50 μl, preferably about 8 to 15 μl, more preferably about 10 nl per eye for up to 4 times per day.
17. An embodiment according to any of the preceding embodiments, wherein the composition is administered to patients treated with first line treatment.
18. An embodiment according to any of the preceding embodiments, wherein the composition is administered to patients treated with steroids as the first line treatment.
19. An embodiment according to embodiment 17 or 18, wherein the inflammatory disease is uveitis.
20. An embodiment according to any of the preceding embodiments the composition comprises 0.03% w/v of tacrolimus, 1.4% w/w ethanol and the semifluorinated alkane is F4H5.
21. An embodiment according to any of the preceding embodiments, wherein the composition further comprises one or more further excipients.
22. An embodiment according to any of the preceding embodiments, wherein the composition essentially consists of 0.03% (w/v) of tacrolimus, 1.4% (w/w) ethanol and the semifluorinated alkane is F4H5.

### DESCRIPTION OF THE DRAWINGS

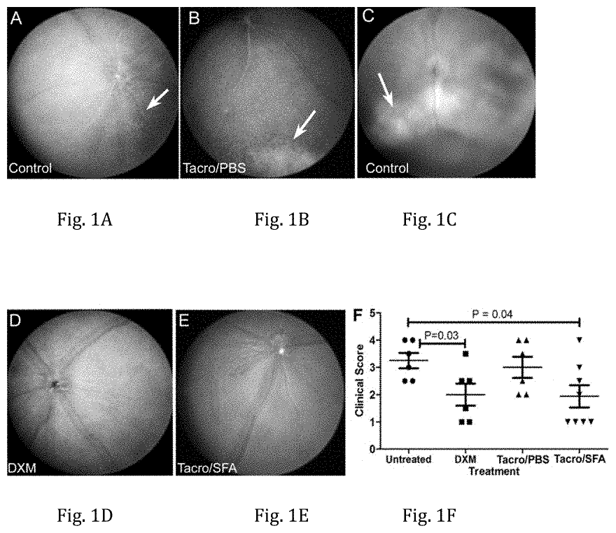

FIGS. 1A to 1F show the effects of tacrolimus/SFA eyedrop treatment on clinical presentations of Endotoxin induced uveitis (EIU) mouse model. EIU was induced in mice by intravitreal injection of 200 ng/μl/eye LPS as described below. Immediately after LPS injection, mice were treated with either 0.1% dexamethasone/PBS (DXM), or 0.03% tacrolimus/PBS (Tacro/PBS), or 0.03% Tacrolimus/SFA (Tacro/SFA) eyedrop, 3 times per day. Clinical evaluation including fundus images were conducted 48 h thereafter. FIGS. 1A to E show fundus images from untreated (A, C), tacrolimus/PBS treated (C), DXM treated (D) and tacrolimus/SFA treated (E) mice. FIG. 1(F) shows the clinical score of retinal inflammation in the different groups; statistics according to the Mann-Whitney test.

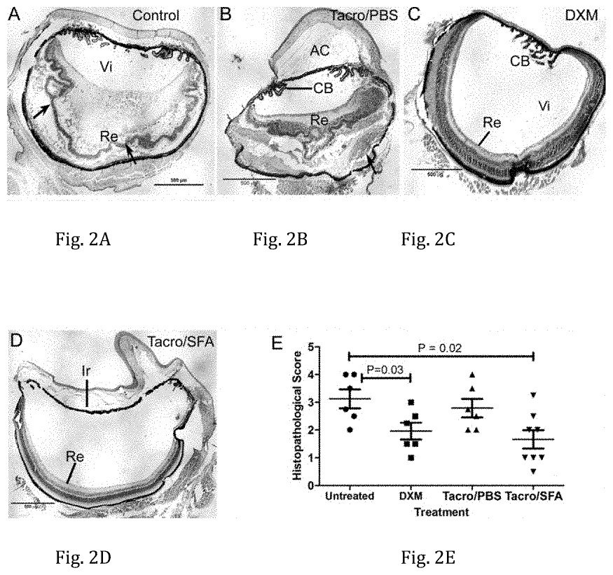

FIGS. 2A to 2E show the histopathology of EIU in different groups of mice. EIU mice were treated with 0.1% dexamethasone/PBS (DXM), or 0.03% Tacrolimus/PBS, or 0.03% tacrolimus/SFA eyedrops 3 times per day from day 0 to day 2 after induction of EIU. Eyes were collected on day 2 and processed for H-E staining (haematoxylin and eosin stain). FIGS. 2A to D show light-microscopic images from untreated EIU mice (A) as control, tacrolimus/PBS treated mice (B), DXM treated mice (C), and tacrolimus/SFA treated mice (D). FIG. 2E shows the histopathological score of retinal inflammation. One plot represents the score of one mouse (i.e. the average score of two eyes of a mouse); statistics according to the Mann-Whitney test (“AC” means anterior chamber; “CB” means ciliary body; “Ir” means iris; “Vi” means vitreous; Re means retina).

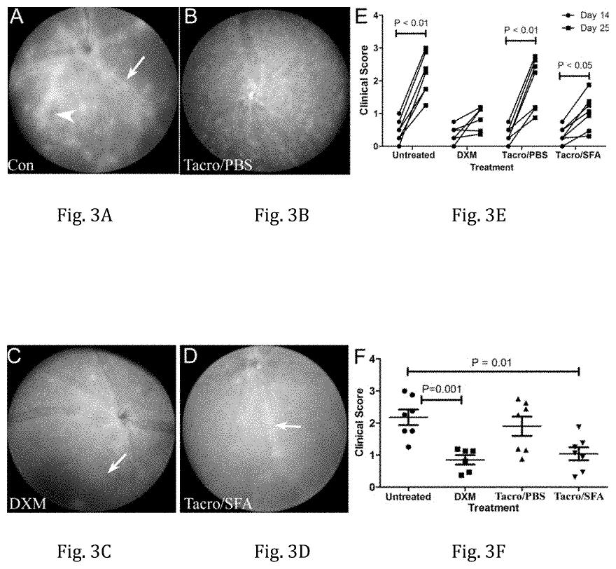

FIGS. 3A to 3F show the effects of tacrolimus/SFA eyedrop treatment on clinical presentation after Experimentally autoimmune uveoretinitis (EAU). EAU was induced in C57BL/6J mice using IRBP₁₋₂₀ peptide immunization. On day 14 post-immunization (p.i.), mice were treated with different eyedrops 3 times per day. Fundus images were taken on day 25 p.i. from a control untreated EAU mouse (A), 0.03% tacrolimus/PBS treated mice (B), 0.1% dexamethasone (DXM) treated mice (C), and 0.03% tacrolimus/SFA treated mice(D). FIG. 3E shows the changes in clinical score of EAU from day 14 p.i. to day 25 p.i. in different groups (statistics according to Wilcoxon matched pairs test). FIG. 3F shows a comparison of clinical score of EAU on day 25 p.i. in the different groups (statistics according to Mann-Whitney test).

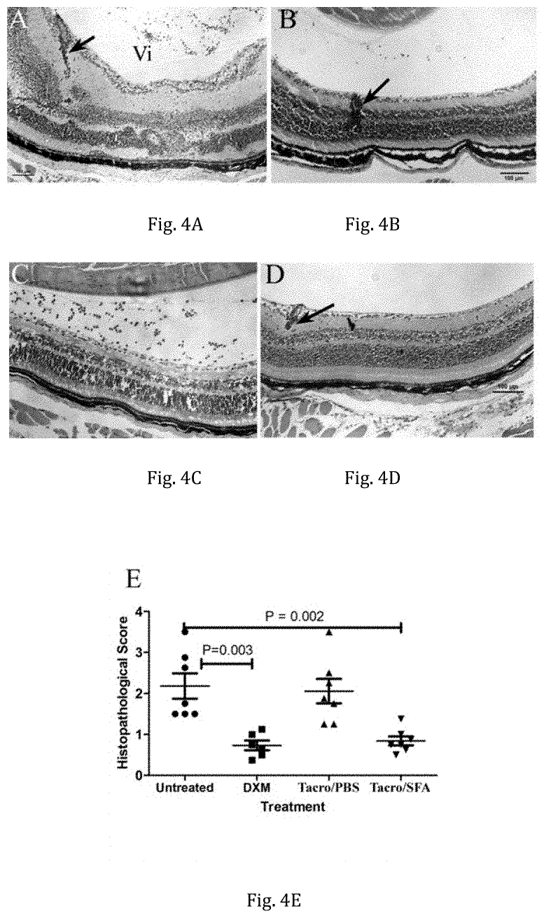

FIGS. 4A to 4E show the histopathology of EAU in different groups of mice. EAU mice were treated with 0.1% dexamethasone (DXM), or 0.03% tacrolimus/SFA eyedrops 3 times per day from day 14 to day 24 p.i. Eyes were collected on day 25 p.i. and processed for H-E staining. FIGS. 4A to D show light-microscopic images of control untreated EAU mice(A), dexamethasone (DXM) treated mice (B), Tacrolimus/PBS treated mice (c), and tacrolimus/SFA treated mice (D). FIG. 4E shows the histopathological score of retinal inflammation. One plot represents the score of one mouse (i.e., the average score of two eyes of a mouse) (statistics according to Mann-Whitney test).

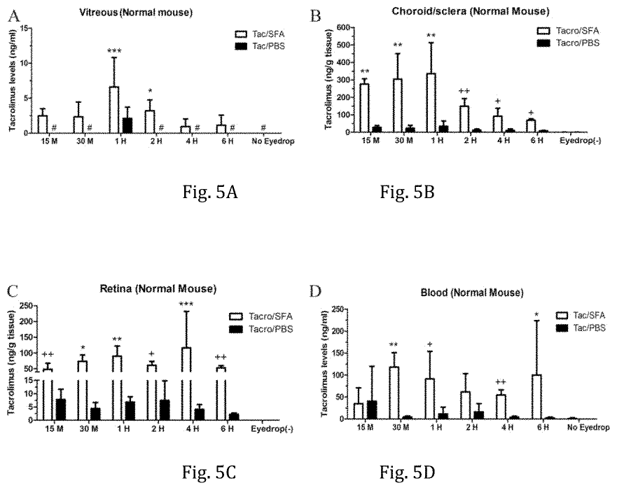

FIGS. 5A to 5D show tacrolimus levels in the vitreous (FIG. 5A), choroid/sclera (FIG. 5B), retina (FIG. 5C) and whole blood (FIG. 5D) of normal mouse eyes. Mice were treated with tacrolimus/SFA or tacrolimus/PBS eyedrop 3 times per day for 3 days. At different times after the last eyedrop treatment (at 15 min, 30 min, 1 h, 2 h, 4 h, 6 h) samples were collected and processed for measurement of tacrolimus using the liquid chromatography tandem mass spectrometry (LC-MS/MS) method. The level of tacrolimus was normalized to tissue weight in the retina and the choroid/sclera. Mean±SD. N=4, * P<0.05, **P<0.01, ***P<0.001 compared to tacrolimus/PBS treated eyes at the same time point, 2-way ANOVA. +P<0.05, ++P<0.01 compared to tacrolimus/PBS treated eyes at the same time point (unpaired t test). “#” means values below detectability in assay.

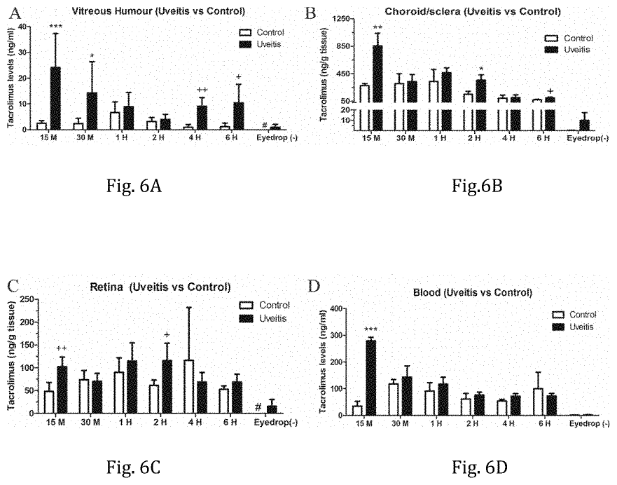

FIGS. 6A to 6D show tacrolimus levels in the vitreous of mouse eyes with and without uveitis. Control non-uveitis mice and uveitis mice were treated with tacrolimus/SFA eyedrop 3 times per day for 3 days. At different times after the last eyedrop treatment (15 min, 30 min, 1 h, 2 h, 4 h, 6 h) samples were collected. FIG. 6A shows tacrolimus levels in the vitreous. FIG. 6B shows tacrolimus levels in the choroid/sclera tissues. FIG. 6C shows tacrolimus levels in the retinal tissues and FIG. 6D shows tacrolimus levels in whole blood. The level of tacrolimus was normalized to the tissue weight in the retina and the choroid/sclera. Mean±SD. N=4; *P<0.05, **P<0.01, ***P<0.001 compared to control non-uveitis eyes at the same time point. 2-way ANOVA.+P<0.05, ++P<0.01 compared to non-uveitis eyes at the same time point (unpaired t test); “#” designate tacrolimus level below detectable value in assay.

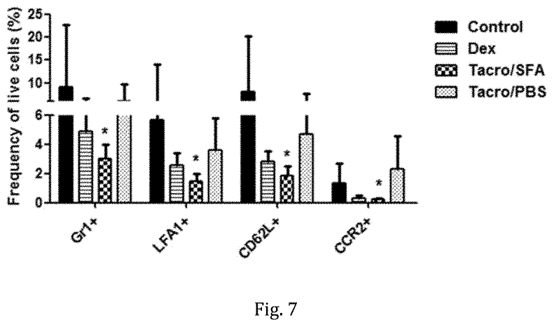

FIG. 7 shows the immune cell infiltration observed in the Endotoxin induced uveitis (EIU) mouse model. Herein, the infiltrating immune cells in retina were measured using flow cytometry. Lower level of infiltrating immune cells were observed in the retina when utilizing 0.03% (w/v) tacrolimus/SFA eyedrops. (control=untreated, Dex=0.1% dexamethasone in PBS, Tacro/SFA=tacrolimus SFA, Tacro/PBS=0.1% tacrolimus in PBS; Infiltrating immune cell markers: Gr1+=granulocytic marker positive cells; LFA1+=Lymphocyte function-associated antigen 1 found on all T-cells and also on B-cells, macrophages and neutrophils; CD62L+=L-selectin (CD62L) positive cell adhesion molecule found on lymphocytes; CCR2+=C-C chemokine receptor type 2 positive inflammatory monocytes)

The following examples serve to illustrate the invention however these are not to be understood as restricting the scope of the invention.

### EXAMPLES

### General:

### Compositions Used:

A solution of 0.03% w/v of tacrolimus [0.3 mg/ml] dissolved in F4H5 comprising 1.4% (w/w) ethanol was formulated to incorporate and deliver the highly lipophilic drug to be administered topically in the form of eye drops to be applied 3 times per day (1 drop/eye). This formulation is abbreviated herein as “Tacro/SFA” or alternatively as “tacrolimus/SFA” or as “Tac/SFA”.

Tacrolimus suspended in PBS (phosphate buffered saline, 0.03%) was used as a control. This formulation is abbreviated herein as “Tacro/PBS” or alternatively as “tacrolimus/PBS” or as “Tac/PBS”.

In addition, 0.1% dexamethasone (DXM) (Sigma-Aldrich, UK) suspended in PBS was used as a standard therapy control. This formulation is abbreviated herein as “DXM” or alternatively as “Dex” or as “0.1% dexamethasone/PBS”.

### Animals:

10- to 12-week-old C57BL/6J mice were treated in conformity with the Home Office Regulations for Animal (Scientific Procedures) Act 1986 (UK) and to the Association for Research in Vision and Ophthalmology Statement for the use of Animals in Ophthalmic and Vision Research. All protocols were approved by the competent Ethics Committee.

### Clinical Evaluation of Ocular Inflammation:

Animals were anesthetized by isoflurane inhalation (Vet Tech Solutions Ltd, UK) and pupils dilated using 1% atropine and 2.5% phenylephrine (Minims, Bausch and Lomb, UK). The severity of ocular inflammation was evaluated under the microscope taking into consideration of both anterior and poster inflammation (by fundus examination using the fundus imaging system). The clinical score for anterior uveitis was graded according to a previously defined scoring system (grades 0 to 4) (Goureau et al., 1995, J. Immunol. 154, 6518-6523):
grade 0, no inflammatory reaction;
grade 1, discrete inflammation of the iris and conjunctival vessels;
grade 2, dilation of the iris and conjunctival vessels with moderate flare in the anterior chamber;
grade 3, hyperemia in the iris associated with the Tyndall effect in the anterior chamber; and
grade 4, same clinical signs as for 3 plus the presence of fibrin or synechiae.

To evaluate posterior inflammation, fundus images were taken from each mouse using the Topical Endoscopic Fundus Imaging (TEFI) system as described previously (Xu et al., 2008, Exp. Eye Res. 87, 319-326). Digital images from each eye were analyzed and clinical score was assessed by two independent researchers using a standard grading system developed by us previously (Xu et al., 2008, Exp. Eye Res. 87, 319-326).

### Histology:

Eyes were collected from day 25 after induction of Experimental Autoimmune Uveoretinitis (EAU) as described below or from day 2 after induction of Endotoxin induced uveitis (EIU) mice for histological examination as described below. All eyes were fixed in 2.5% (w/v) glutaraldehyde (Agar Scientific Ltd, Stansted, UK) for at least 24 h. Eyes were then embedded in paraffin and processed for haematoxylin and eosin (H&E) staining. For each eye, four sections from four different layers were graded according to the criteria described previously (Agarwal et al., 2012, Methods Mol. Biol. 900, 443-469).

### Example 1: Endotoxin Induced Uveitis (EIU) Mouse Model

Induction of Endotoxin Induced Uveitis (EIU) as a Model for Human Acute Anterior Uveitis:

EIU was induced in C57BL/6J mice by intravitreal injection of LPS (lipopolysaccharide) using a previously described protocol (Rosenbaum et al., 2011, Invest. Ophthalmol. Vis. Sci. 52, 6472-6477) slightly modified as follows: Escherichia coli 055:B5 LPS (Sigma, UK) was dissolved in pyrogen-free phosphate buffered saline (PBS). Mice were injected intravitreally with 200 ng/eye LPS in 1 μL using a 30-gauge needle and a 25 μL syringe in a Repeating Dispenser (PB600-1, Hamilton, Nev., USA). Intraocular inflammation began 4 to 6 hours after the LPS challenge by intravitreal injection and peaked at 24 to 48 h. The inflammation then started to resolve after 3 days. The eye may return to normal within a week (Rosenbaum et al., 2011, Invest. Ophthalmol. Vis. Sci. 52, 6472-6477).

### Treatment of EIU:

Mice were randomized into four groups in the EIU study: Group 1: Control (no eyedrops), n=6 mice; Group 2: treatment with 0.1% dexamethasone, n=6 mice; Group 3: treatment with 0.03% tacrolimus/PBS, n=6 mice; Group 4: 0.03% tacrolimus/SFA, n=8 mice. All mice were treated with eyedrops three times per day starting immediately after the intravitreal injection of LPS for two days. The study was repeated twice.

### The Effect of Tacrolimus/SFA Eyedrop in the Treatment of EIU:

Clinical investigation revealed engorgement of conjunctival vessels, massive immune cell infiltration in the anterior chamber and the vitreous of untreated and tacrolimus-PBS treated EIU mice at 48 h (see FIGS. 1A & 1B; arrows). Infiltrating cells were more frequently observed in the inferior part of the vitreous body (FIG. 1B). Retinal detachment and haemorrhage were observed in eyes with severe inflammation (FIG. 1C). The severity of intraocular inflammation was reduced following dexamethasone (FIG. 1D) or tacrolimus/SFA (FIG. 1E) treatment compared to control and Tacrolimus/PBS treated groups (FIG. 1F).

Consistent with the clinical signs, histological analysis revealed marked features of EIU characterized by accumulation of immune cells in the anterior chamber (AC), vitreous cavity (Vi), and cilliary body (CB) area in mice with no treatment (FIG. 2A) or Tacrolimus/PBS eyedrop treatment (FIG. 2B). Severe retinal destruction (retinal detachment, disorganized retinal layers, haemorrhage) associated with massive retinal immune cell infiltration was also observed (arrows in FIGS. 2A & 2B). Mild cell infiltration in the vitreous and retina was observed in eyes treated with 0.1% dexamethasone or tacrolimus/SFA eyedrops, and the overall architecture of retinal layers remained intact (FIGS. 2C & 2D). The histopathological scores in dexamethasone and tacrolimus/SFA treated eyes were significantly lower than that from untreated control EIU eyes (FIG. 2E). Repeated studies in additional two sets of mice revealed similar results.

In summary, treatment with tacrolimus/SFA eyedrops reduced the severity of acute uveitis in the Endotoxin induced uveitis (EIU) mouse model: Reductions were observed in the clinical score as well as in the histological score. Furthermore, a lower level of infiltrating immune cells in the retina (located in the back of the eye) was observed.

### Example 2: Experimentally Autoimmune Uveoretinitis (EAU)

Induction of Experimental Autoimmune Uveoretinitis (EAU) Mouse Model for Posterior Uveitis:

EAU was induced as previously described by Chen et al., 2012 and Xu et al., 2005. CS7BL/61 mice were immunized subcutaneously with 500 μg of IRPB peptide 1-20 (GPTHLFQPSLVLDMAKVLLD; GL (Biochem) Shanghai Ltd, China) emulsified in complete Freund's adjuvant (CFA, H37Ra, Difco Laboratories, Detroit, Mich., USA). Mice were administered with an additional intraperitoneal injection of 100 μ1 (1.5 μg) of Bordetella pertussis toxin (Tocris Bioscience, UK). Retinal inflammation developed at day 12 to 14 post-immunisation (p.i.), and peaked at day 22 to 25 p.i. The severity of inflammation declines after the peak stage, however, retinal inflammation remains active for over 4 months (Chen et al., 2012).

### Treatment of EAU:

On day 14 p.i. EAU mice were divided into four groups based on the clinical score of inflammation and the score in each group was comparable. Group 1: Control (no treatment, n=7 mice); Group 2: 0.1% dexamethasone (DXM) eyedrops (n=7 mice); Group 3: 0.03% tacrolimus/PBS eyedrops (n=7 mice); Group 4: 0.03% tacrolimus/SFA eyedrops (n=7 mice). All mice in groups 2 to 4 received eyedrop treatment three times a day from day 14 p.i. to day 25 p.i.

The Therapeutic Role of Tacrolimus/SFA Eyedrop in the Mouse Model of Posterior Uveitis (EAU):

Having shown that treatment with tacrolimus/SFA eyedrops suppressed inflammation in EIU, its therapeutic effects were further evaluated in EAU, a mouse model of posterior uveitis. Topical administration of eyedrops was started after the onset of uveitis i.e., 14 days p.i.. Clinical scores of EAU were comparable between study groups prior to treatment (i.e., day 14). Severe retinal inflammation, characterized by extensive retinal infiltration (whitish lesions, see FIGS. 3A & 3B), vascular cuffing (arrows, FIG. 3A), and linear lesions (arrowhead, FIG. 3A) was observed in untreated and tacrolimus/PBS treated EAU mice. Mild inflammation characterized by discrete small infiltrates, mild vascular cuffing and optic head swelling (FIGS. 3C & 3D) was observed in dexamethasone and tacrolimus/SFA treated eyes. The severity of retinal inflammation increased from day 14 to day 25 p.i. in all groups, although the increment was more significant in untreated and tacrolimus/PBS treated groups compared to tacrolimus/SFA and dexamethasone treated groups (FIG. 3E). On day 25 p.i., the clinical scores of the dexamethasone and tacrolimus/SFA treated groups were significantly lower than those from untreated mice (FIG. 3F).

Histological examination revealed massive immune cell infiltration in the retina and vitreous in eyes from untreated (FIG. 4A) and tacrolimus/PBS treated (FIG. 4C) mice. Retinal layers were disorganized in these eyes. Small numbers of immune cell infiltration and granulomatous lesions (arrows, FIGS. 4A to 4D) were observed in dexamethasone (FIG. 4B) and Tacrolimus/SFA (FIG. 4D) treated eyes, although the retinal structure was largely intact in these mice. The overall histopathological score of EAU was significantly lower in dexamethasone treated and tacrolimus/SFA treated mice compared to untreated controls (FIG. 4E). Our results suggest that Tacrolimus/SFA eyedrop is effective for the control of ongoing ocular inflammation in EAU.

In Summary, Treatment with Tacrolimus/SFA Eyedrops Reduced Retinal Inflammation in the Experimental Autoimmune Uveoretinitis (EAU) Mouse Model:

Reductions were observed in the clinical score as well as in the histological score.

### Example 3: Pharmacokinetics and Absorption Study of Tacrolimus/SFA Eyedrop

Eyedrop Treatment and Groups:

The pharmacokinetics of tacrolimus/SFA eyedrop were conducted in both normal mice and EIU mice (as described above). Normal C57BL/6J mice or EIU mice (immediately after LPS injection) were treated with either 0.03% tacrolimus/SFA or 0.03% tacrolimus/PBS eyedrops (60 μL/drop) three times per day for three days. At different times (15 min, 30 min, 1 h, 2 h, 4 h and 6 h) after the last eyedrop treatment, animals were sacrificed and the following samples were collected for the measurement of tacrolimus (1) vitreous humor, (2) retina, (3) choroid/sclera, and (4) blood. 4 mice were used in each group, and 20 normal mice and 8 EIU mice that did not receive any eyedrop treatment were used as controls.

Sample Collection and Processing:

6 to 8 μl of vitreous humor were collected from each mouse. Retinal tissues were dissected and weighted immediately after sacrificing the animal and put into an Eppendorf tube. Choroid/scleral tissues were dissected and weighted immediately after sacrificing the animal and put into an Eppendorf tube. 200 to 500 μl of whole blood were collected into EDTA-coated tubes from each mouse.

All samples were stored at −20° C. and then tacrolimus was quantitatively determined by liquid chromatography tandem mass spectrometry (LC-MS/MS) method, which was qualified for the respective matrices before sample analysis. The lower limit of quantification was 0.25 ng/ml for blood, 0.05 ng/ml for retina and choroid/sclera homogenate, and 1.25 ng/ml for the vitreous.

### Pharmacokinetics and Absorption of Tacrolimus Eyedrop in Normal Non-Inflamed Mice:

The levels of tacrolimus in the vitreous humor collected at 15 min to 2 h after tacrolimus/SFA eyedrop treatment were between 2 to 6 ng/ml. By 4 h and 6 h, the level of tacrolimus was below detectable level in most of the samples in tacrolimus/SFA treated mice. In tacrolimus/PBS treated mice the tacrolimus level was below the detectable level in all samples apart from those from 1 h. The level of tacrolimus in the vitreous from tacrolimus/SFA group was significantly higher than those from tacrolimus/PBS group at 1h after administration (FIG. 5A).

In the choroid/sclera, high levels of tacrolimus were detected at 15 min to 1 h after tacrolimus/SFA eyedrop administration (276 ng/g tissue to 337 ng/g tissue, FIG. 5B). The levels then reduced to 150 ng/g tissue at 2 h and 70 ng/g tissue at 6 h (FIG. 5B). In eyes treated with tacrolimus/PBS, 23 ng/g and 34 ng/gram tissue of tacrolimus were detected between 15 min and 1 h, and the levels reduced to 8.45 ng/g tissue by 6 h (FIG. 5B). The levels of tacrolimus in the choroid/sclera in the tacrolimus/SFA group at all time points were significantly higher than those in tacrolimus/PBS group (FIG. 5B).

The levels of tacrolimus in the retina in the tacrolimus/SFA were 48 ng/g tissue at 15 min after eyedrop administration, and increased to 90 ng/g tissue by 1 h (FIG. 5C). The levels of tacrolimus decreased slightly after 2 h, but remained at 53 ng/g tissue at 6 h after eyedrop treatment (FIG. 5C). In eyes treated with tacrolimus/PBS, the levels of tacrolimus were between 2 to 7 ng/g tissue at all time points and were significantly lower than those in eyes treated with tacrolimus/SFA (FIG. 5C).

The level of tacrolimus in the blood of normal mice treated with tacrolimus/SFA increased from 35 ng/ml at 15 min to 118 ng/ml at 30 min and then reduced from 1 h but remained at 62 ng/ml at 2 h after treatment (FIG. 5D). Lower level of tacrolimus (2 to 40 ng/ml) was detected in the blood in mice treated with tacrolimus/PBS eyedrop. There was no difference between tacrolimus/PBS treated and untreated controls (FIG. 5D). The levels of tacrolimus in the blood in tacrolimus/SFA group were significantly higher than those in tacrolimus/PBS group at 30 min, 1 h and 4 h time points (FIG. 5D).

The results show that tacrolimus/SFA has a greater permeability than tacrolimus/PBS, and can rapidly penetrate ocular barriers in normal mouse eyes and distribute to all ocular tissues as well as the blood circulation rapidly (within 15 to 30 min) after eyedrop administration.

### Pharmacokinetics and Absorption of Tacrolimus Eyedrop in Uveitis Mice:

As only tacrolimus/SFA was tested in the uveitis eyes, the pharmacokinetics of tacrolimus/SFA eyedrop between uveitis eyes and non-uveitis eyes have been compared. The levels of tacrolimus in the vitreous of uveitis mice were 24 ng/ml and 14 ng/ml at 15 min and 30 min respectively, significantly higher than those in non-uveitis mice (2.5 ng/ml and 2.3 ng/ml respectively). Six hours after eyedrop administration 10 ng/ml of Tacrolimus were detected in the vitreous of uveitis eyes compared to 1.1 ng/ml in non-uveitis eyes (FIG. 6A).

The level of tacrolimus in the choroid/sclera was 855.5 ng/g tissue at 15 min after eyedrop administration in uveitis mice compared to 276 ng/g tissue in normal mice (FIG. 6B). The concentrations of tacrolimus decreased at 30 min and 1 h in uveitis eyes to the levels that were comparable to non-uveitis eye. At 2 h and 6 h, uveitis mice had significantly higher levels of tacrolimus compared to normal non-inflamed mice (FIG. 6B).

Significantly higher levels of tacrolimus were detected in the retina of uveitis mice compared to that of non-uveitis mice after 15 min (102 vs 48 ng/g tissue) and 2 h (115 ng/g vs 61 ng/g tissue) (FIG. 6C) of administration. The level of retinal tacrolimus at 6 h was approximately 67% of that in 15 min in uveitis eyes (FIG. 6C).

The blood level of tacrolimus was 280 ng/ml in uveitis mice 15 min after administration, significantly higher than that in non-uveitis mice (35 ng/ml) (FIG. 6D). The levels then decreased in uveitis mice to 143 ng/ml at 30 min and to 73 ng/ml at 6 h after eyedrop treatment. In non-inflamed mice, the blood levels of tacrolimus increased at 30 min to 118 ng/ml, and remained at the levels comparable to uveitis mice at other time points (FIG. 6D).

The present pharmacokinetic study shows that tacrolimus/SFA, but not tacrolimus/PBS rapidly (within 15 minutes) penetrates the tissue barriers (e.g., epithelial, membranes and endothelial barriers) after topical administration and reaches ocular tissues such as choroid/sclera at significant levels in normal mouse.

The penetration into the ocular tissues (such as choroid/sclera and retina) is much increased when the eye is inflamed, and the levels of tacrolimus in the choroid/sclera of uveitis mice were 3 and 8 times higher than those in non-uveitis mice 15 min after administration.

Furthermore, tacrolimus/SFA penetrates not only the general tissue barrier, but also the ocular barrier. Significant amounts tacrolimus were detected in the vitreous and retina after topical administration at 15 min and the drug remained in the retina for at least 6 h in normal mouse eyes. The levels of tacrolimus in the vitreous and retina were 2 and 10 times higher in uveitis eye than those in non-uveitis eyes at 15 min after administration.

The data suggest that tacrolimus/SFA is able to penetrate ocular barriers (such as choroid/sclera) in mice, reach intraocular tissue (such as retina) at therapeutic levels and suppress retinal inflammation. It is shown that 0.03% tacrolimus/SFA eyedrops suppress intraocular inflammation in both EIU and EAU models. Tacrolimus/PBS did not show any suppressive effect in EIU and EAU models and it was unable to penetrate ocular barriers.

1. A method of treating an intraocular inflammatory eye disease comprising administering to a patient in need thereof a pharmaceutical composition comprising a therapeutically effective amount of tacrolimus and a liquid vehicle comprising at least one semifluorinated alkane.

2. The method according to claim 1, wherein the intraocular inflammatory eye disease is selected from the group consisting of uveitis, retinal inflammation, scleritis and optic neuritis or combinations thereof.

3. The method according to claim 1, wherein the intraocular inflammatory eye disease is selected from uveitis, retinal inflammation and combinations thereof.

4. The method according to claim 3, wherein the intraocular inflammatory eye disease is uveitis.

5. The method according to claim 1, wherein the uveitis is anterior uveitis and/or intermediate uveitis and/or posterior uveitis.

6. The method according to claim 1, wherein the semifluorinated alkane is a compound of the formula F(CF₂)ₙ(CH₂)ₘH wherein n and m are integers independently selected from the range of 3 to 10.

7. The method according to claim 1, wherein said semifluorinated alkane is selected from the group consisting of F4H5, F4H6, F6H6 and F6H8.

8. The method according to claim 1, wherein said composition is formulated as a solution, suspension or emulsion.

9. The method according to claim 1, wherein said composition is topically administered to the eye, eye lid, eye sac, eye surface or to an ophthalmic tissue.

10. The method according to claim 1, wherein said composition is administered as single drops with a volume of about 5 to 50 μl per dose per eye.

11. The method according to claim 1, wherein said composition is administered up to 4 times per day.

12. The method according to claim 1, wherein the composition is administered to patients treated with first line treatment of an inflammatory eye disease.

13. The method according to claim 1, wherein the composition is administered to patients treated with steroids as the first-line treatment.

14. The method according to claim 1, wherein the composition comprises 0.01% (w/v) to 0.05% (w/v) of tacrolimus, 0.2 to 1.4% (w/w) ethanol and the semifluorinated alkane is F4H5.

15. A kit comprising the pharmaceutical composition according to claim 16 and a container for holding the composition, wherein the container comprises a dispensing means adapted for topical administration of the composition to an eye surface.

16. A pharmaceutical composition comprising a therapeutically effective amount of tacrolimus and a liquid vehicle comprising at least one semifluorinated alkane, wherein the semifluorinated alkane is selected from the group consisting of F4H5, F4H6, F6H6 and F6H8, and the composition the comprises 0.01% (w/v) to 0.05% (w/v) of tacrolimus, and 0.2 to 1.4% (w/w) ethanol.

17. The composition according to claim 16, wherein the semifluorinated alkane is F4H5.

18. The kit according to claim 15, wherein the dispensing means is adapted for topical administration of the composition to a lower eyelid, to the lacrimal sac or to an ophthalmic tissue.

19. The method according to claim 1, wherein the intraocular inflammatory eye disease is retinal inflammation, scleritis, optic neuritis, or a combination thereof.

20. The method according to claim 1, wherein the intraocular inflammatory eye disease is scleritis, optic neuritis, or a combination thereof.
